(19) United States
(12) Patent Application Publication
(10) Pub. No.: US 2021/0402000 A1
(43) Pub. Date: Dec. 30, 2021

(54) HIGH-POLYMER-DENSITY BIOCONJUGATE COMPOSITIONS AND RELATED METHODS

(71) Applicant: UNIVERSITY OF WASHINGTON, Seattle, WA (US)

(72) Inventors: Shaoyi Jiang, Seattle, WA (US); Zhefan Yuan, Seattle, WA (US); Liqian Niu, Seattle, WA (US)

(73) Assignee: UNIVERSITY OF WASHINGTON, Seattle, WA (US)

(21) Appl. No.: 17/283,731

(22) PCT Filed: Oct. 10, 2019
(86) PCT No.: PCT/US2019/055577

§ 371 Date: Apr. 8, 2021

Related U.S. Application Data
(60) Provisional application No. 62/743,670, filed on Oct. 10, 2018.

(51) Int. Cl. A61K 47/64; A61K 47/59

(52) U.S. Cl. CPC A61K 47/6425; A61K 47/593

(57) ABSTRACT

High-polymer-density bioconjugate compositions including multi-layer polymer bioconjugates, polymer backfilled bioconjugates, and multi-layer polymer backfilled bioconjugates, and methods for making the compositions.

### CROSS-REFERENCE TO RELATED APPLICATION

This application claims the benefit of U.S. Patent Application No. 62/743,670, filed Oct. 10, 2018, expressly incorporated herein by reference in its entirety.

### STATEMENT OF GOVERNMENT LICENSE RIGHTS

This invention was made with Government support under Grant No. HDTRA1-13-1-0044 awarded by the Defense Threat Reduction Agency under Grant No. R21 EB027843 awarded by the National Institute of Health. The Government has certain rights in the invention.

### BACKGROUND OF THE INVENTION

Naturally derived biologic agents comprise a broad reservoir of drug candidates with exceptional medicinal potency and specificity. Many biotherapeutic regimens such as enzyme replacement therapies require multiple administrations to fully cure the disease. However, repeated injections of biomolecule drugs with high immunogenicity also increase the risk of immune responses in patients, which could deprive them of life-sustaining therapies and even cause fatal adverse reactions. Basically, the undesirable immune responses toward foreign proteins attenuate their therapeutic effect mainly through the generation of antigen-specific antibody (Ab). Once Ab binds to biomolecule drugs, it would directly neutralize their pharmacological activity (neutralizing) or diminish their therapeutic exposure (non-neutralizing) via accelerated blood clearance (ABC). The Ab against the biomolecule itself, which is usually referred to the antidrug Abs (ADA), is generated mainly due the low protecting polymer density on protein surfaces.

Some biomolecules intrinsically have limited conjugation sites, making high-polymer-density bioconjugates very hard to achieve or impossible using conventional conjugation technologies. For example, organophosphorus hydrolase (OPH), a bacterial originated enzyme, shows strong catalytic efficacy to organophosphorus pesticides and nerve agents. There are only four (4) water accessible lysine groups per OPH monomer that are accessible for modification with polymers. Lysine-targeted polymer conjugation technology like PEGylation cannot fully cover OPH with sufficient protection due to the lack of conjugation sites available. In other cases, during a polymer conjugation process, initially conjugated polymers will block the conjugation of subsequent polymers due to steric hindrance.

The present invention seeks to provide advanced conjugation technologies that are effective in increasing the number of polymers covalently coupled to a biomolecule or for improving the efficiency of polymer conjugation to a biomolecule to provide in high-polymer-density bioconjugate compositions.

### SUMMARY OF THE INVENTION

The present invention provides high-polymer-density bioconjugate compositions and methods for making the compositions.

In one aspect, the invention provides multi-layer polymer bioconjugates. In one embodiment, the bioconjugate comprises a biomolecule having first polymers covalently coupled the biomolecule and second polymers covalently coupled to at least a portion of the first polymers.

The invention provides methods for making multi-layer bioconjugates. In one embodiment, the method comprises:

(a) covalently coupling first polymers to a biomolecule to provide a biomolecule having a first polymer layer (i.e., inner polymer layer) surrounding the biomolecule; and

(b) covalently coupling second polymers to at least a portion of the first polymers of the first polymer layer to provide a biomolecule having a second polymer layer (i.e., outer polymer layer) surrounding the biomolecule (i.e., a multi-layer bioconjugate).

The invention provides methods for increasing the number of reactive groups (e.g., amine) in a biomolecule. In one embodiment, the method comprises covalently coupling first polymers to a biomolecule to provide a biomolecule having a first polymer layer (i.e., inner polymer layer) surrounding the biomolecule, wherein the first polymers comprise from 2 to about 1000 reactive groups.

In another aspect, the invention provides polymer backfilled bioconjugates. In one embodiment, the polymer backfilled bioconjugate comprises:

(a) biomolecule having one or more first reactive groups and one or more second reactive groups, wherein the first and second reactive groups are different;

(b) one or more first polymers covalently coupled to the first reactive groups; and

(c) one or more second polymers (i.e., backfilled polymers) covalently coupled to the second reactive groups.

The invention provides methods for making polymer backfilled bioconjugates. In one embodiment, the method comprises:

(a) covalently coupling one or more first polymers to a biomolecule having one or more first reactive groups and one or more second reactive groups, wherein the first and second reactive groups are different, and wherein the first polymers are covalently coupled to the first reactive groups; and

(b) covalently coupling one or more second polymers to the second reactive groups to provide a bioconjugate having first polymers and second polymers covalently coupled to the biomolecule.

The invention provides methods for increasing the number of polymers covalently coupled to a biomolecule. In one embodiment, the method comprises:

(a) covalently coupling first polymers to a biomolecule having one or more first reactive groups and one or more second reactive groups, wherein the first and second reactive groups are different, and wherein the first polymers are covalently coupled to the first reactive groups; and

(b) covalently coupling one or more second polymers to the second reactive groups to provide a bioconjugate having first polymers and second polymers covalently coupled to the biomolecule, thereby increasing the density of polymers covalently coupled to the biomolecule.

In a further aspect, the invention provides multi-layer/polymer backfilled bioconjugates. In one embodiment, multi-layer/polymer backfilled bioconjugate comprises:

(a) biomolecule having one or more first reactive groups and one or more second reactive groups, wherein the first and second reactive groups are different;

(b) first polymers covalently coupled to the first reactive groups;

(c) second polymers covalently coupled to at least a portion of the first polymers and;

(d) one or more third polymers (i.e., backfilled polymers) covalently coupled to the second reactive groups.

The invention provides methods for making multi-layer/polymer backfilled bioconjugates. In one embodiment, the method comprises:

(a) covalently coupling one or more first polymers to a biomolecule having one or more first reactive groups and one or more second reactive groups, wherein the first and second reactive groups are different, and wherein the first polymers are covalently coupled to the first reactive groups;

(b) covalently coupling one or more second polymers to at least a portion of the first polymers; and

(c) covalently coupling one or more third polymers to the second reactive groups to provide a bioconjugate having first polymers, second polymers, and third polymers covalently coupled to the biomolecule.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a schematic illustration of the preparation of a representative peptide of the invention, (EK)₁₀-C peptide.

FIG. 2A is a schematic illustration of the preparation of representative peptide conjugates of the invention (EK-asparaginase conjugates): ASP-EK-S (single layer conjugate), ASP-EK-D (double layer conjugate), and ASP-EK-T (triple layer conjugate).

FIG. 2B is a gel permeation chromatogram (GPC) comparing the size distribution of representative peptide conjugates of the invention to native asparaginase (ASP): ASP-EK-S (single layer conjugate), ASP-EK-D (double layer conjugate, and ASP-EK-T (triple layer conjugate).

FIG. 3 compares in vitro anti-asparaginase antibody binding affinity of native ASP, and representative peptide conjugates of the invention, ASP-EK-S, ASP-EK-D, and ASP-EK-T. The original concentration was 1 μmol/mL.

FIGS. 4A-4D compare Hydrophobic Interaction Chromatography (HIC) results for native ASP (4A), and representative peptide conjugates of the invention, ASP-EK-S (4B), ASP-EK-D (4C), and ASP-EK-T (4D).

FIGS. 5A-5C compare pharmacokinetic profiles of a representative peptide conjugates of the invention, ASP-EK-T, relative to native ASP as a function of dose: first dose (5A), second dose (5B), and third dose (5C).

FIG. 6 compares the antibody titers for native ASP and ASP-EK-T: native ASP anti-ASP, ASP-EK-T anti-ASP, and ASP-EK-T anti-EK.

FIG. 7 is a gel permeation chromatogram (GPC) comparing the size distribution of native asparaginase (ASP) with representative peptide conjugates of the invention: ASP-EK-PCB (single EK layer).

FIG. 8 is a schematic illustration the preparation of a representative peptide conjugate of the invention: a polymer backfilled conjugate (PCB-OPH).

FIG. 9 is a gel permeation chromatogram (GPC) comparing the size distribution of native organophosphorous hydrolase (OPH) with representative peptide conjugates of the invention: PCB-OPH with backfill and PCB-OPH without backfill.

FIGS. 10A-10C compare pharmacokinetic profiles of free OPH (10A) with representative peptide conjugates of the invention: PCB-OPH without backfill (10B) and PCB-OPH with backfill (10C).

FIGS. 11A and 11B compare anti-OPH IgG (11A) and anti-OPH IgM (11B) titers in a rat model for native OPH and representative peptide conjugates of the invention: PCB-OPH with backfill and PCB-OPH without backfill.

FIG. 12 is a gel permeation chromatogram (GPC) comparing the size distribution of native OPH with representative polymer conjugates of the invention: OPH-EK, OPH-PCB, OPH-EK-PCB without backfill, and OPH-EK-PCB with backfill.

### DETAILED DESCRIPTION OF THE INVENTION

The present invention provides high-polymer-density bioconjugate compositions and methods for making the compositions. The high-polymer-density bioconjugate compositions are characterized as a biomolecule that is advantageously surrounded (e.g., covered) with one or more polymers that impart super-low immunogenicity to the bioconjugate while at the same time preserving the functionality of the native biomolecule.

In one aspect, multi-layer polymer bioconjugates are provided. In another aspect polymer backfilled bioconjugates are provided. In a further aspect, multi-layer polymer backfilled bioconjugates are provided.

These high-polymer-density bioconjugates are useful developing biotherapeutics, treating or preventing diseases, disorders, or conditions, and otherwise improving a subject's health or wellbeing.

### Multi-layer Polymer Bioconjugates

In one aspect, the invention provides multi-layer polymer bioconjugates. In one embodiment, the bioconjugate comprises a biomolecule having first polymers covalently coupled the biomolecule and second polymers covalently coupled to at least a portion of the first polymers.

The first polymers form a first polymer layer (i.e., an inner polymer layer) that surrounds (or substantially surrounds) the biomolecule and the second polymers form a second polymer layer (i.e., an outer polymer layer) that surrounds (or substantially surrounds) the first polymer layer and the biomolecule.

In certain embodiments, the first polymers form a first layer surrounding the biomolecule and the second polymers form a second layer surrounding the biomolecule.

Biomolecules that are advantageously modified to provide the multi-layer polymer bioconjugates of the invention, include proteins, glycoproteins, proteoglycans, lipids, nucleic acids, cells, viruses, or bacteria. Representative biomolecules are described in detail below.

In certain embodiments, the first polymers are zwitterionic polymers or peptides. In certain embodiments, the second polymers are zwitterionic polymers or peptides.

In certain of these embodiments, the peptides are EK-containing peptides. As used herein, the term “EK-containing peptide” refers to a peptide having substantially the same number of E (glutamic acid) and K (lysine) residues (e.g., from about 2 to about 100 EK residues). In certain embodiments, the EK-containing peptide is an (EK)ₙ peptide, where n is from 1 to about 50. In certain of these embodiments, n is 2 to about 50. In other embodiments, n is 3 to about 50. In further embodiments, n is 4 to about 50. In other embodiments, n is 5 to about 50. In further embodiments, n is 1 to 10.

It will be appreciated that an EK-containing peptide may further include one or more additional peptide residues, such as a proline, glycine, serine, threonine, glutamine, asparagine residues (e.g., an EKX-containing peptide, where X is P, G, S, T, Q, or N). Representative EKX-containing peptides include EKP and EKG. In certain EKX-containing peptides, the ratio of E:K is 1:1 (or substantially 1:1) and the ratio of E:K:X is 1:1:n, where n is 1 or a fraction from 0.1 to 1.

In other of these embodiments, the peptide is an unstructured recombinant polypeptide (URP). In certain of these embodiments, the URP comprising at least 40 contiguous amino acids, wherein (a) the sum of glycine (G), aspartate (D), alanine (A), serine (S), threonine (T), glutamate (E) and proline (P) residues contained in the URP, constitutes at least 80% of the total amino acids of the URP, and the remainder, when present, consists of arginine or lysine, and the remainder does not contain methionine, cysteine, asparagine, and glutamine; (b) wherein said URP comprises at least three different types of amino acids; and (c) at least 50% of the at least 40 contiguous amino acids in said URP are devoid of secondary structure as determined by Chou-Fasman algorithm. Unstructured recombinant polypeptides are described in U.S. Pat. No. 7,855,279, expressly incorporated herein by reference in its entirety.

In further of these other embodiments, the peptide is a random coil polypeptide. In certain of these embodiments, the random coil polypeptide comprises 50 to 3000 amino acids and consisting solely of proline and alanine, wherein the polypeptide forms a random coil. In certain embodiments, the random coil polypeptide consists of 10% to 75% proline residues. In other embodiments, the random coil polypeptide comprises a plurality of amino acid repeats wherein no more than 6 consecutive amino acid residues are the same amino acid. Random coil polypeptides are described in U.S. Pat. No. 9,221,882, expressly incorporated herein by reference in its entirety.

In certain embodiments of the bioconjugate, the first polymers are EK-containing peptides and the second polymers are EK-containing peptides or zwitterionic polymers.

The first polymers include reactive groups for available for further covalent coupling, such as to second polymers. In certain embodiments, the first polymers (i.e., the inner polymer layer) include from 2 to about 1000 reactive groups. As used herein, the term “reactive groups” refers to functional groups that are capable of covalent coupling by chemical conjugation methods. Suitable reactive groups include amino (—NH₂) groups, such as the ε-amino group of a lysine residue, and carboxylic acid (—CO₂H) or carboxylate (—CO₂) groups. For example, for an EK-containing peptide that is (EK)ₙ, where n is 1 to 50, for n=1 there are two (2) amine groups and 1 carboxylic acid group for a total of three (3) reactive groups, and for n=50 there are fifty-one (51) amine groups and fifty (50) carboxylic acid groups, for a total of one hundred and one (101) reactive groups.

In certain embodiments, the first polymers are peptides that include one or more amino acid residues selected from lysine, glutamic acid, aspartic acid, cysteine, histidine, serine, threonine, tyrosine, tryptophan, and proline residues. In certain of these embodiments, the first polymers are peptides that include one or more lysine residues.

In other embodiments, the first polymers are non-peptides polymers that include one or more functional groups selected from amine, carboxylic acid, thiol, maleimide, carbon-carbon double bond, carbon-carbon triple bond, and azido functional groups.

The second polymers include reactive groups for available for further covalent coupling, such as to first polymers. In certain embodiments, the second polymers (i.e., the outer polymer layer) comprises from 1 to about 1000 reactive groups. Representative reactive groups include functional groups such as amine, carboxylic acid (carboxylate), thiol, maleimide, carbon-carbon double bond, carbon-carbon triple bond, and azido functional groups.

In certain embodiments, the second polymers are peptides that include one or more amino acid residues selected from lysine, glutamic acid, aspartic acid, cysteine, histidine, serine, threonine, tyrosine, tryptophan, and proline residues. In certain of these embodiments, the second polymers are peptides that include one or more lysine or glutamic acid residues. In certain of these embodiments, the second polymers are EK-containing polymers, as described herein.

In other embodiments, the second polymers are non-peptides polymers that are water soluble. Representative second polymers that are hydrophilic and having water solubility include non-peptides polymers, such as poly(carboxybetaine) (PCB), poly(sulfobetaine) (PSB), poly(2-methacryloyloxyethyl phosphorylcholine) (PMPC), poly(tetramethylamine oxide) (TMAO), poly(2-oxazoline) (POZ), poly(N-(2-hydroxypropyl)methacrylamide) (polyHPMA), and polyethylene glycol (PEG) polymers.

In certain embodiments, the second polymer is a zwitterionic polymer, such as a poly(carboxybetaine) (PCB), poly(sulfobetaine) (PSB), poly(2-methacryloyloxyethyl phosphorylcholine) (PMPC), and poly(tetramethylamine oxide) (TMAO) polymer. In one embodiment, the second polymers are poly(carboxybetaine) (PCB) polymers.

In other embodiments, the second polymer is an unstructured recombinant polypeptide (URP) or a random coil polypeptide, as described herein.

In addition to the inner polymer layer (first polymers) and outer polymer layer (second polymers), the multi-layer bioconjugate may include one or more additional polymer layers (e.g., 1-10 layers) intermediate the first layer and the second layer.

In certain embodiments, the composition of each of the additional polymer layers is the same as the compositions of the first polymer layers described herein.

In certain embodiments, the bioconjugate includes three layers, where the first two layers (i.e., inner polymer layers) are derived from the same first polymers (e.g., EK-containing peptides) and the third layer (i.e., outer polymer layer) is derived the same polymer (e.g. EK-containing peptide) or a different polymer (e.g., zwitterionic polymer, such as PCB, or a unstructured recombinant polypeptide or a random coil polypeptide.

Methods for Making Multi-Layer Bioconjugates

In another aspect, the invention provides methods for making multi-layer bioconjugates. In one embodiment, the method comprises:

(a) covalently coupling first polymers to a biomolecule to provide a biomolecule having a first polymer layer (i.e., inner polymer layer) surrounding the biomolecule; and

(b) covalently coupling second polymers to at least a portion of the first polymers of the first polymer layer to provide a biomolecule having a second polymer layer (i.e., outer polymer layer) surrounding the biomolecule (i.e., a multi-layer bioconjugate).

Method for Increasing Reactive Group Number in a Biomolecule

In further aspect, the invention provides methods for increasing the number of reactive groups (e.g., amine) in a biomolecule. In one embodiment, the method comprises covalently coupling first polymers to a biomolecule to provide a biomolecule having a first polymer layer (i.e., inner polymer layer) surrounding the biomolecule, wherein the first polymers comprise from 2 to about 1000 reactive groups.

In certain embodiments, using an (EK)ₙ peptide (n=1-50) as the first polymer the method increase the number of reactive groups by 2 to 51 amine groups or 3-101 including amine and carboxylic acid (carboxylate) groups.

In certain embodiments, the method further comprises covalently coupling second polymers to at least a portion of the first polymers of the first polymer layer to provide a biomolecule having a second polymer layer (i.e., outer polymer layer) surrounding the biomolecule (i.e., a multi-layer bioconjugate composition), wherein the second polymers include from 1 to about 1000 reactive groups. In certain of these embodiments, the second polymers comprise from 1 to about 50 reactive groups.

The preparation and characterization of representative multi-layer polymer bioconjugates is described in Examples 1, 2, and 3.

The following is a further description of the multi-layer polymer bioconjugate of the invention.

As used herein, the term “multi-layer polymer bioconjugate” refers to a bioconjugate having a multi-layer structure polymer surrounding or substantially surrounding a biomolecule. In certain embodiments, the number of layers of multi-layer polymer ranges from 2 to 10, 2 to 8, 2 to 6, 2 to 4 and 2 to 3, preferable number is from 2 to 3. In some embodiments, a biomolecule is covalently conjugated by a multi-layer structure polymer. In some embodiments, a biomolecule is covalently conjugated by multiple multi-layer structure polymers. In some embodiments, polymers are grafted from a biomolecule to form multi-layer structure. In some embodiments, multi-layer structure polymers are pre-built up and then grafted to a biomolecule.

Bioconjugate Inner Layer

In certain embodiments, the term “inner layer” of the multi-layer polymer is any layer between the outer polymer and the biomolecule. In certain embodiments, the number of inner layers ranges from 1 to 10, 1 to 8, 1 to 6, 1 to 4, and 1 to 2. In certain embodiments, the inner polymer has 2 to about 1000 reactive groups. At least 1 reactive group is connecting to one of reactive group from biomolecule and the others are conjugation sites for next inner layer polymer or the outer layer polymer. The conjugation of inner layer polymer provides more conjugation sites for following layer polymer attachment.

In some embodiments, the first polymer (i.e., inner polymer) is hydrophilic peptide, comprising reactive amino acid residues selected from lysine, glutamic acid, aspartic acid, cysteine, histidine, serine, threonine, tyrosine and tryptophan. In some embodiments, hydrophilic peptide contains lysine, glutamic acid, aspartic acid and cysteine. In some embodiments, particularly preferred hydrophilic peptide contains lysine, glutamic acid and cysteine.

In some embodiments, the inner polymer is hydrophilic non-peptide polymer, comprising reactive groups selected from amine, carboxylic acid, thiol, carbon-carbon double bond, carbon-carbon triple bond and azido groups. In some embodiments, particularly preferred hydrophilic non-peptide polymer contains amine, carboxyl acid and thiol groups.

Examples of hydrophilic inner layer polymers include amine containing polymers having amine groups on either the polymer backbone or the polymer side chains, such as poly-L-lysine and other polyamino acids of natural or synthetic amino acids or mixtures of amino acids, including poly(lysine-co-glutamic acid), poly(lysine-co-aspartic acid), poly(D-lysine), poly(ornithine), poly(arginine), and poly(histidine), and nonpeptide polyamines such as poly(aminostyrene), poly(aminoacrylate), poly(N-methyl aminoacrylate), poly(N-ethylaminoacrylate), poly(N,N-dimethyl aminoacrylate), poly(N,N-diethylaminoacrylate), poly(aminomethacrylate), poly(N-methyl aminomethacrylate), poly(N-ethyl aminomethacrylate), poly(N,N-dimethyl aminomethacrylate), poly(N,N-diethyl aminomethacrylate), poly(ethyleneimine), polymer of quaternary amines, such as poly(N,N,N-trimethylaminoacrylate chloride), poly(methyacrylaminopropyltrimethyl ammonium chloride) and their derivatives. Examples of inner polymers also include neutral polymers derivatives from synthetic polymers such as poly(oxazoline), poly(N-vinyl pyrrolidone), and poly(amino acids), such as poly(serine), poly(threonine), and poly(glutamine).

Bioconjugate Outer Layer

The second polymer (i.e., outer polymer) layer polymer protects the biomolecule and enlarges the hydrodynamic size of the whole bioconjugate. In certain embodiments, the outer layer polymer has 1 to 1000 reactive groups. The outer layer polymer has at least 1 reactive group to connect with inner layer polymer.

In some embodiments, outer layer polymer has only 1 reactive group to connect with inner polymer. After the connection, outer layer polymer cannot be further modified or connected with other small molecules or polymers.

In some embodiments, the outer polymer is hydrophilic peptide, comprising reactive amino acid residues selected from lysine, glutamic acid, aspartic acid, cysteine, histidine, serine, threonine, tyrosine and tryptophan. In some embodiments, hydrophilic peptide contains lysine, glutamic acid, aspartic acid and cysteine. In some embodiments, particularly preferred hydrophilic peptide contains lysine, glutamic acid and cysteine.

In some embodiments, the outer polymer is hydrophilic non-peptide polymer, comprising reactive groups selected from amine, carboxylic acid, thiol, double bond, triple bond and azido groups. In some embodiments, particularly preferred reactive groups are amine or thiol groups. As defined herein, “hydrophilic polymers” are those which are soluble in water or mixtures of water and some polar organic solvents, such as low molecular weight alcohols, acetone, dimethylformamide, dimethyl sulfoxide, dioxane, acetonitrile and tetrahydrofuran. The polar organic solvent is preferably present at a concentration of about 0 to 50% by volume.

As used herein, “water soluble” means that the entire polymer must be completely soluble in aqueous or aqueous/organic solutions, such as buffered saline or buffered saline with small amounts of added organic solvents as co-solvents.

Examples of water-soluble polymers include polyamines having amine groups on either the polymer backbone or the polymer side chains, such as poly-L-lysine and other polyamino acids of natural or synthetic amino acids or mixtures of amino acids, including poly(lysine-co-glutamic acid), poly(lysine-co-aspartic acid), poly(D-lysine), poly(ornithine), poly(arginine), and poly(histidine), and nonpeptide polyamines such as poly(aminostyrene), poly(aminoacrylate), poly(N-methyl aminoacrylate), poly(N-ethylaminoacrylate), poly(N,N-dimethyl aminoacrylate), poly(N,N-diethylaminoacrylate), poly(aminomethacrylate), poly(N-methyl aminomethacrylate), poly(N-ethyl aminomethacrylate), poly(N,N-dimethyl aminomethacrylate), poly(N,N-diethyl aminomethacrylate), poly(ethyleneimine), polymers of quaternary amines, such as poly(N,N,N-trimethylaminoacrylate chloride), poly(methyacrylaminopropyltrimethyl ammonium chloride), poly(ethyloxazoline), poly(N-vinyl pyrrolidone), and neutral poly(amino acids) such as poly(serine), poly(threonine), and poly(glutamine).

Suitable outer layer polymers include neutral and negatively charged polymers. Preferred outer layer polymers include neutral polymers.

Other suitable polymers include naturally occurring proteins, such as gelatin, bovine serum albumin, and ovalbumin, as well as complex sugars, such as hyaluronic acid, starches and agarose. The polymer can be any biocompatible water-soluble polyelectrolyte polymer.

Hydrophilic polymers also include poly(oxyalkylene oxides) such as poly(ethylene oxide), poly(vinyl alcohol), natural or synthetic polysaccharides and polysaccharide derivatives such as alginate, chitosan, dextran, water soluble cellulose derivatives such as hydroxy ethyl cellulose and carboxymethylcellulose, poly(hydroxyethyl acrylate), poly(hydroxy ethylmethacrylate), and polyacrylamides such as isopropylacrylamide. As used herein “derivatives” include polymers having substitutions, additions of chemical groups, for example, alkyl, alkylene, hydroxylations, oxidations and other modifications routinely made by those skilled in the art.

In some embodiments, preferred outer layer hydrophilic polymer are poly(ethylene glycol) (PEG), poly(carboxybetaine) (PCB), poly(2-methacryloyloxylethyl phosphorylcholine) (PMPC), poly(sulfobetaine) (PSBMA), poly(2-oxazolines) (POZ), and poly(N-(2-hydroxypropyl)methacrylamide) (polyHPMA).

Other outer polymers include unstructured recombinant polypeptides and random coil polypeptides, as described herein.

Bioconjugate Biomolecules

In certain embodiments, the biomolecule of the multi-layer polymer bioconjugate is a protein, a peptide, a nucleic acid, a virus, a glycoprotein, a proteoglycan, or a lipid.

Proteins or peptides. In some embodiments, a biomolecule is a protein or a peptide, including an enzyme, a cytokine, a hormone, a growth factor, an antigen, an antibody, a characteristic portion of an antibody, a clotting factor, a regulatory protein, a signaling protein, a transcription protein, and a receptor. These may include IL-1 α, IL-1 β, IL-2, IL-3, IL-4, IL-5, IL-6, IL-11, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-31, IL-32, IL-33, colony stimulating factor-1 (CSF-1), macrophage colony stimulating factor, glucocerobrosidase, thyrotropin, stem cell factor, granulocyte macrophage colony stimulating factor, granulocyte colony stimulating factor (G-CSF), GM-CSF, (EOS)-CSF, CSF-1, EPO, organophosphorus hydrolase (OPH), interferon-alpha (IFN-α), consensus interferon-beta (IFN-β), interferon-gamma (IFN-γ), thrombopoietin (TPO), Cas9, Cas12a, Cas12b, Cas12c, Cas13a1, Cas13a2, Cas13b, Angiopoietin-1 (Ang-1), Ang-2, Ang-4, Ang-Y, angiopoietin-like polypeptide 1 (ANGPTL1), angiopoietin-like polypeptide 2 (ANGPTL2), angiopoietin-like polypeptide 3 (ANGPTL3), angiopoietin-like polypeptide 4 (ANGPTL4), angiopoietin-like polypeptide 5 (ANGPTL5), angiopoietin-like polypeptide 6 (ANGPTL6), angiopoietin-like polypeptide 7 (ANGPTL7), vitronectin, vascular endothelial growth factor (VEGF), angiogenin, activin A, activin B, activin C, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, bone morphogenic protein receptor II, brain derived neurotrophic factor, cardiotrophin-1, ciliary neutrophic factor, ciliary neutrophic factor receptor, cripto, cryptic, cytokine-induced neutrophil chemotactic factor 1, cytokine-induced neutrophil, chemotactic factor 2α, hepatitis B vaccine, hepatitis C vaccine, drotrecogin .alpha., cytokine-induced neutrophil chemotactic factor 2β, SLF, SCF, mast cell growth factor, endothelial cell growth factor, endothelin 1, epidermal growth factor (EGF), epigen, epiregulin, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor 11, fibroblast growth factor 12, fibroblast growth factor 13, fibroblast growth factor 16, fibroblast growth factor 17, fibroblast growth factor 19, fibroblast growth factor 20, fibroblast growth factor 21, fibroblast growth factor acidic, fibroblast growth factor basic, EPA, Lactoferrin, H-subunit ferritin, prostaglandin (PG) E1 and E2, glial cell line-derived neutrophic factor receptor α1, glial cell line-derived neutrophic factor receptor, growth related protein, growth related protein a, IgG, IgE, IgM, IgA, and IgD, α-galactosidase, β-galactosidase, DNAse, fetuin, leutinizing hormone, alteplase, estrogen, insulin, albumin, lipoproteins, fetoprotein, transferrin, thrombopoietin, urokinase, integrin, thrombin, Factor IX (FIX), Factor VIII (FVIII), Factor Vila (FVIIa), Von Willebrand Factor (VWF), Factor FV (FV), Factor X (FX), Factor XI (FXI), Factor XII (FXII), Factor XIII (FXIII), thrombin (FII), protein C, protein S, tPA, PAI-1, tissue factor (TF), ADAMTS 13 protease, growth related protein β, growth related protein, heparin binding epidermal growth factor, hepatocyte growth factor, hepatocyte growth factor receptor, hepatoma-derived growth factor, insulin-like growth factor I, insulin-like growth factor receptor, insulin-like growth factor II, insulin-like growth factor binding protein, keratinocyte growth factor, leukemia inhibitory factor, somatropin, antihemophiliac factor, pegaspargase, orthoclone OKT 3, adenosine deaminase, alglucerase, imiglucerase, leukemia inhibitory factor receptor α, nerve growth factor nerve growth factor receptor, neuropoietin, neurotrophin-3, neurotrophin-4, oncostatin M (OSM), placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor (SCF), stem cell factor receptor, TNF, TNF0, TNF1, TNF2, transforming growth factor α, hymic stromal lymphopoietin (TSLP), tumor necrosis factor receptor type I, tumor necrosis factor receptor type II, urokinase-type plasminogen activator receptor, phospholipase-activating protein (PUP), insulin, lectin ricin, prolactin, chorionic gonadotropin, follicle-stimulating hormone, thyroid-stimulating hormone, tissue plasminogen activator (tPA), leptin, Enbrel (etanercept), activin, inhibin, leukemic inhibitory factor, oncostatin M, MIP-1-C, MIP-1 B; MIP-2-C, GRO-C, MIP-2-B, and platelet factor-4.

In some embodiments the functional biomolecule may be another designed functional polypeptide sequence. In some embodiments the functional polypeptide sequence is a domain or fragment of a functional polypeptide. In some embodiments the functional polypeptide sequence is a recognition sequence, which optionally results in stoichiometric binding or modification of the polypeptide. In some embodiments the functional polypeptide sequence is a sequence useful for promoting expression or purification of the fusion polypeptide. In some embodiments the functional polypeptide sequence is a structural motif of a secondary or higher nature, comprising helices, sheets, turns, folds, and super domains. In some embodiments the functional polypeptide sequence is a linker sequence that exists between mixed charge polypeptide and another functional biomolecule.

In some embodiments, the functional biomolecule is a protein, which modified through rational design, directed evolution, or some other technique yielding a functional protein improved in at least one aspect of performance.

The terms “protein,” “peptide,” “functional protein,” and “functional peptide” can be used interchangeably. In certain embodiments, peptides range from about 5 to about 40000, about 5 to about 20000, about 5 to about 10000, about 5 to about 5000, about 5 to about 1000, about 5 to about 750, about 5 to about 500, about 5 to about 250, about 5 to about 100, about 5 to about 75, about 5 to about 50, about 5 to about 40, about 5 to about 30, about 5 to about 25, about 5 to about 20, about 5 to about 15, or about 5 to about 10 amino acids in size.

Nucleic acids. In certain embodiments of the invention, the biomolecule is a nucleic acid (e.g., DNA, RNA, derivatives thereof). In some embodiments, the nucleic acid agent is a functional RNA. In general, a “functional RNA” is an RNA that does not code for a protein but instead belongs to a class of RNA molecules whose members characteristically possess one or more different functions or activities within a cell. It will be appreciated that the relative activities of functional RNA molecules having different sequences may differ and may depend at least in part on the particular cell type in which the RNA is present. Thus, the term “functional RNA” is used herein to refer to a class of RNA molecule and is not intended to imply that all members of the class will in fact display the activity characteristic of that class under any particular set of conditions. In some embodiments, functional RNAs include RNAi-inducing entities (e.g., short interfering RNAs (siRNAs), short hairpin RNAs (shRNAs), and microRNAs), ribozymes, tRNAs, rRNAs, RNAs useful for triple helix formation.

In some embodiments, the nucleic acid agent is a vector. As used herein, the term “vector” refers to a nucleic acid molecule (typically, but not necessarily, a DNA molecule) that can transport another nucleic acid to which it has been linked. A vector can achieve extra-chromosomal replication and/or expression of nucleic acids to which they are linked in a host cell. In some embodiments, a vector can achieve integration into the genome of the host cell.

In some embodiments, vectors are used to direct protein and/or RNA expression. In some embodiments, the protein and/or RNA to be expressed is not normally expressed by the cell. In some embodiments, the protein and/or RNA to be expressed is normally expressed by the cell, but at lower levels than it is expressed when the vector has not been delivered to the cell. In some embodiments, a vector directs expression of any of the functional RNAs described herein, such as RNAi-inducing entities, ribozymes.

Viruses. In some embodiments, the biomolecule is a virus. In certain embodiments, viruses have utility in medical therapy and diagnosis in medical and veterinary practice and in agriculture. They are of particular use in gene therapy (for example the delivery of genes for the localized expression of a desired gene product) and for non-gene therapy applications such as, but without limitation, viral oncolysis. In certain embodiments, virus is selected from the following families and groups: Adenoviridae; Bimaviridae; Bunyaviridae; Caliciviridae; Capillovirus group; Carlavirus group; Carmovirus virus group; Group Caulimovirus; Closterovirus Group; Commelina yellow mottle virus group; Comovirus virus group; Coronaviridae; PM2 phage group: Corcicoviridae; Group Cryptic virus; group Cryptovirus; Cucumovirus virus group Family φ6 phage group; Cystoviridae; Group Carnation ringspot; Dianthovirus virus group; Group Broad bean wilt; Fabavirus virus group; Filoviridae; Flaviviridae; Furovirus group; Group Geminivirus; Group Giardiavirus; Hepadnaviridae; Herpesviridae; Hordeivirus virus group; liarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae including adeno-associated viruses; Paramyxoviridae; Parsnip yellow fleck virus group; Partitiviridae; Parvoviridae; Pea enation mosaic virus group; Phycodnaviridae; Picomaviridae; Plasmaviridae; Podoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites. In certain embodiments, particularly preferred viruses for the purpose of delivery of transgenes include, for example, retrovirus, adenovirus, adenoassociated virus, herpesvirus and poxvirus. Adenovirus and adeno-associated virus are particularly preferred.

Glycoproteins and proteoglycans. In some embodiments, the biomolecule is glycoprotein or proteoglycan, which is a carbohydrate associated with a protein. A glycoprotein or proteoglycan may be natural or synthetic. A carbohydrate may also be a derivatized natural carbohydrate. In certain embodiments, a carbohydrate may be a simple or complex sugar. In certain embodiments, a carbohydrate is a monosaccharide, including but not limited to glucose, fructose, galactose, and ribose. In certain embodiments, a carbohydrate is a disaccharide, including but not limited to lactose, sucrose, maltose, trehalose, and cellobiose. In certain embodiments, a carbohydrate is a polysaccharide, including, but not limited to, cellulose, microcrystalline cellulose, hydroxypropyl methylcellulose (HPMC), methylcellulose (MC), dextrose, dextran, glycogen, xanthan gum, gellan gum, starch, and pullulan. In certain embodiments, a carbohydrate is a sugar alcohol, including but not limited to mannitol, sorbitol, xylitol, erythritol, malitol, and lactitol.

Lipids. In some embodiments, the biomolecule is a lipid. In certain embodiments, the lipid is a lipid that is associated with a protein (e.g., lipoprotein). Exemplary lipids that may be used in accordance with the present invention include, but are not limited to, oils, fatty acids, saturated fatty acid, unsaturated fatty acids, essential fatty acids, cis fatty acids, trans fatty acids, glycerides, monoglycerides, diglycerides, triglycerides, hormones, steroids (e.g., cholesterol, bile acids), vitamins (e.g., vitamin E), phospholipids, sphingolipids, and lipoproteins.

In some embodiments, the lipid may comprise one or more fatty acid groups or salts thereof. In some embodiments, the fatty acid group may comprise digestible, long chain (e.g., C8-C50), substituted or unsubstituted hydrocarbons. In some embodiments, the fatty acid group may be one or more of butyric, caproic, caprylic, capric, lauric, myristic, palmitic, stearic, arachidic, behenic, or lignoceric acid. In some embodiments, the fatty acid group may be one or more of palmitoleic, oleic, vaccenic, linoleic, alphalinolenic, gamma-linoleic, arachidonic, gadoleic, arachidonic, eicosapentaenoic, docosahexaenoic, or erucic acid.

### Polymer Backfilled Bioconjugates

In another aspect, the invention provides polymer backfilled bioconjugates. In one embodiment, the polymer backfilled bioconjugate comprises:

(a) biomolecule having one or more first reactive groups and one or more second reactive groups, wherein the first and second reactive groups are different;

(b) one or more first polymers covalently coupled to the first reactive groups; and

(c) one or more second polymers (i.e., backfilled polymers) covalently coupled to the second reactive groups.

In the polymer backfilled bioconjugates, the first polymers are covalently coupled the biomolecule by reaction of a suitable functional group on the first polymers and the biomolecule's first reactive groups. The second polymers then backfill the bioconjugate: the second polymers are covalently coupled the biomolecule by reaction of a suitable functional group on the second polymers and the biomolecule's second reactive groups.

In certain embodiments, the first reactive groups are selected from amine, carboxylic acid (carboxylate), thiol, maleimide, carbon-carbon double bond, carbon-carbon triple bond, and azido groups. In other embodiments, the first reactive groups are selected from amine, carboxylic acid (carboxylate), thiol, and maleimide groups. In further embodiments, the first reactive groups are amine groups.

In certain embodiments, the second reactive groups are selected from amine, carboxylic acid (carboxylate), thiol, maleimide, carbon-carbon double bond, carbon-carbon triple bond, and azido groups. In other embodiments, the second reactive groups are selected from amine, carboxylic acid (carboxylate), thiol, and maleimide groups. In further embodiments, the second reactive groups are amine groups.

In certain embodiments, the first reactive groups are selected from amine groups and the second reactive groups are selected from carboxylic acid (or carboxylate) groups.

In one embodiment of the polymer backfilled bioconjugates, the first polymers and second polymers are hydrophilic, substantially water-soluble polymers selected from poly(carboxybetaine) (PCB), poly(sulfobetaine) (PSB), poly(2-methacryloyloxyethyl phosphorylcholine) (PMPC), poly(tetramethylamine oxide) (TMAO), poly(2-oxazoline) (POZ), poly(N-(2-hydroxypropyl)methacrylamide) (polyHPMA), and polyethylene glycol (PEG) polymers.

In other embodiments, the first and/or second polymers are EK-containing peptides.

In further embodiments, the first and/or second polymers are unstructured recombinant polypeptides or random coil polypeptides, as noted above and described in U.S. Pat. Nos. 7,855,279 and 9,221,882.

In another embodiment, the first polymers and second polymers are zwitterionic polymers selected from poly(carboxybetaine) (PCB), poly(sulfobetaine) (PSB), poly(2-methacryloyloxyethyl phosphorylcholine) (PMPC), and poly(tetramethylamine oxide) (TMAO) polymers.

In certain of the above embodiments of the polymer backfilled bioconjugates polymer, first polymers and the second polymers are the same (e.g., PCB/PCB, PSB/PSB, PC/PC, and PTMAO/PTMAO). In other of the above embodiments, the first polymers and the second polymers are the different (e.g., PCB/PSB, PSB/PCB, PCB/PC, PSB/PC, PC/PCB, and PC/PCB).

In a further embodiment, the first polymers are poly(carboxybetaine) (PCB) polymers and the second polymers are poly(carboxybetaine) (PCB) polymers.

Biomolecules that are advantageously modified to provide the backfilled polymer bioconjugates of the invention, include proteins, glycoproteins, proteoglycans, lipids, nucleic acids, cells, viruses, or bacteria. Representative biomolecules are described in detail above.

The preparation and characterization of a representative polymer backfilled bioconjugate is described in Example 4.

Methods for Making Polymer Backfilled Bioconjugates

In another aspect, the invention provides methods for making polymer backfilled bioconjugates. In one embodiment, the method comprises:

(a) covalently coupling one or more first polymers to a biomolecule having one or more first reactive groups and one or more second reactive groups, wherein the first and second reactive groups are different, and wherein the first polymers are covalently coupled to the first reactive groups; and

(b) covalently coupling one or more second polymers to the second reactive groups to provide a bioconjugate having first polymers and second polymers covalently coupled to the biomolecule.

Method for Increasing the Number of Polymers Coupled to a Biomolecule

In a further aspect, the invention provides methods for increasing the number of polymers covalently coupled to a biomolecule. In one embodiment, the method comprises:
(a) covalently coupling first polymers to a biomolecule having one or more first reactive groups and one or more second reactive groups, wherein the first and second reactive groups are different, and wherein the first polymers are covalently coupled to the first reactive groups; and
(b) covalently coupling one or more second polymers to the second reactive groups to provide a bioconjugate having first polymers and second polymers covalently coupled to the biomolecule, thereby increasing the density of polymers covalently coupled to the biomolecule.

Multi-Layer/Polymer Backfilled Bioconjugates

In a further aspect, the invention provides multi-layer/polymer backfilled bioconjugates. In one embodiment, multi-layer/polymer backfilled bioconjugate comprises:

(a) biomolecule having one or more first reactive groups and one or more second reactive groups, wherein the first and second reactive groups are different;

(b) first polymers covalently coupled to the first reactive groups;

(c) second polymers covalently coupled to at least a portion of the first polymers and;

(d) one or more third polymers (i.e., backfilled polymers) covalently coupled to the second reactive groups.

In certain embodiments, the first and second polymers are the same as the first and second polymers, as described above for the multi-layer polymer bioconjugates. In certain embodiments, the first and/or second polymers are zwitterionic polymers or peptides (e.g., EK-containing peptides, such as (EK)ₙ peptides where n is from 1 to 50).

In other embodiments, the first and/or second polymers are EK-containing peptides.

In further embodiments, the first and/or second polymers are unstructured recombinant polypeptides or random coil polypeptides, as noted above and described in U.S. Pat. Nos. 7,855,279 and 9,221,882.

In certain embodiments, the third polymers are polymers selected from the group consisting of poly(carboxybetaine) (PCB), poly(sulfobetaine) (PSB), poly(2-methacryloyloxyethyl phosphorylcholine) (PMPC), poly(tetramethylamine oxide) (TMAO), poly(2-oxazoline) (POZ), poly(N-(2-hydroxypropyl)methacrylamide) (polyHPMA), and polyethylene glycol (PEG) polymers.

Biomolecules that are advantageously modified to provide the backfilled polymer bioconjugates of the invention, include proteins, glycoproteins, proteoglycans, lipids, nucleic acids, cells, viruses, or bacteria. Representative biomolecules are described in detail above.

Methods for Making Multi-Layer/Polymer Backfilled Bioconjugates

In another aspect, the invention provides methods for making multi-layer/polymer backfilled bioconjugates. In one embodiment, the method comprises:

(a) covalently coupling one or more first polymers to a biomolecule having one or more first reactive groups and one or more second reactive groups, wherein the first and second reactive groups are different, and wherein the first polymers are covalently coupled to the first reactive groups;

(b) covalently coupling one or more second polymers to at least a portion of the first polymers; and

(c) covalently coupling one or more third polymers to the second reactive groups to provide a bioconjugate having first polymers, second polymers, and third polymers covalently coupled to the biomolecule.

The preparation of a representative multi-layer/polymer backfilled bioconjugate is described in Example 5.

As used herein, the term “about” refers to ±5% of the value specified.

The following examples are provided for the purpose of illustrating, not limiting, the invention.

### EXAMPLES

### Example 1

The Preparation and Characterization of Representative Multi-Layer Polymer Bioconjugates: Asp-EK-S, Asp-EK-D, and Asp-EK-T

In this example, the preparation and characterization of representative multi-layer polymer bioconjugates, Asp-EK-S, Asp-EK-D, and Asp-EK-T, is described. The high polymer density is achieved by multiple lysine amplifications.

Different proteins show different surface residues geo-distribution. Protein needs abundant accessible surface groups to provide enough conjugation sites. Usually, amine groups from lysine are chosen as reactive group to connect with polymers. Whether the target protein containing enough surface lysines, the lysine amplification technology described herein provides solutions for obtaining high polymer density on target proteins.

Synthesis and Characterization of (EK)₁₀-C Polypeptide

(EK)₁₀-C was synthesized by Fmoc Solid Phase Peptide Synthesis (SPPS) on Liberty Blue Automated Microwave Assisted Peptide Synthesizer (CEM). Sequence synthesis scale was set at 2.5 mmol on Rink amide MBHA resin (0.6 meq/g substitution). Deprotection was performed in 20% piperidine/DMF solution with machine default microwave conditions. Coupling reactions were performed in the presence of a 5-fold molar excess of reagents [0.2 M amino acid solution (in DMF) with 0.5M DIC (in DMF) and 1.0 M Oxyma (in DMF)] by using 2.5 mmol coupling cycle method provided from CEM. Cleavage was performed using 20 ml of cocktail (TFA/phenol/water/thioanisole/EDT; 82.5/5/5/5/2.5) for 180 min at room temperature. Following cleavage, (EK)₁₀-C—NH₂ was precipitated out and washed with ice-cold anhydrous ethyl ether. Econo-Pac 10DG Desalting Columns were used to further enhance the purity of the products.

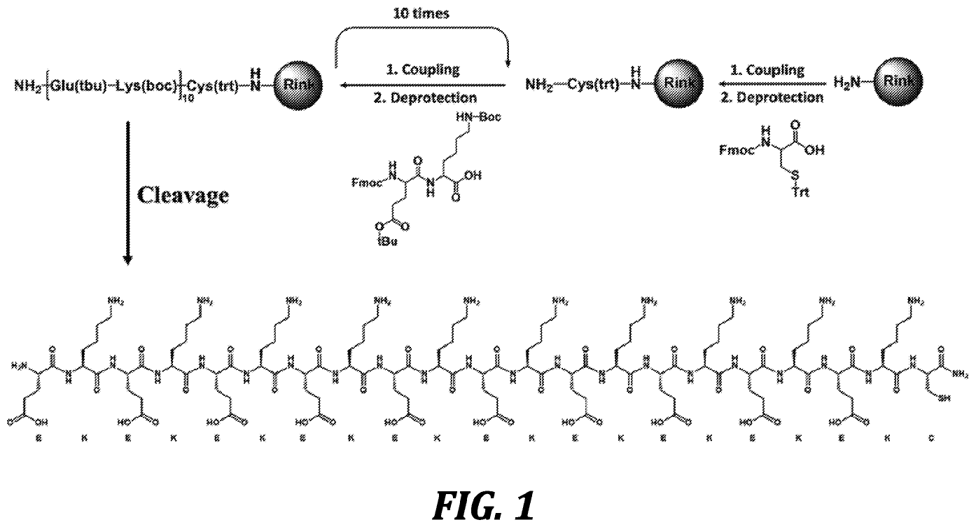

The principle of the synthesis process is shown in FIG. 1.

Synthesis of Multi-Layer Polypeptide-Asparaginase Conjugate

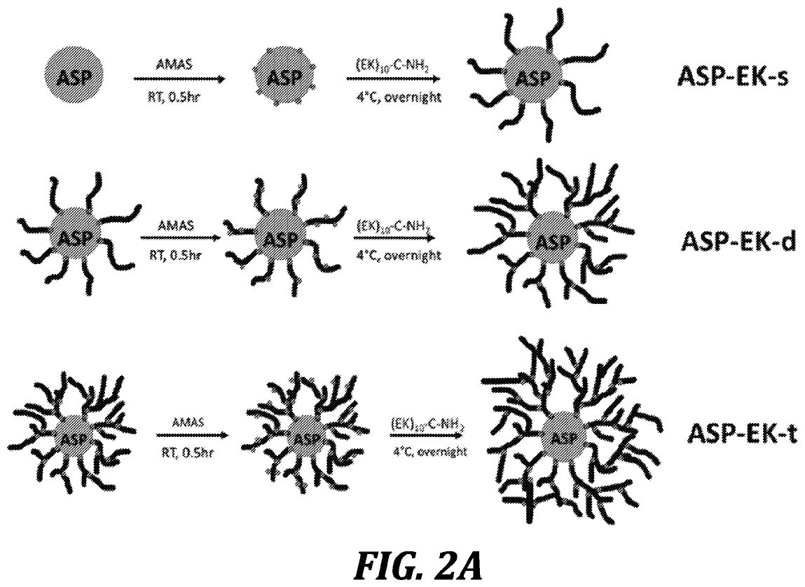

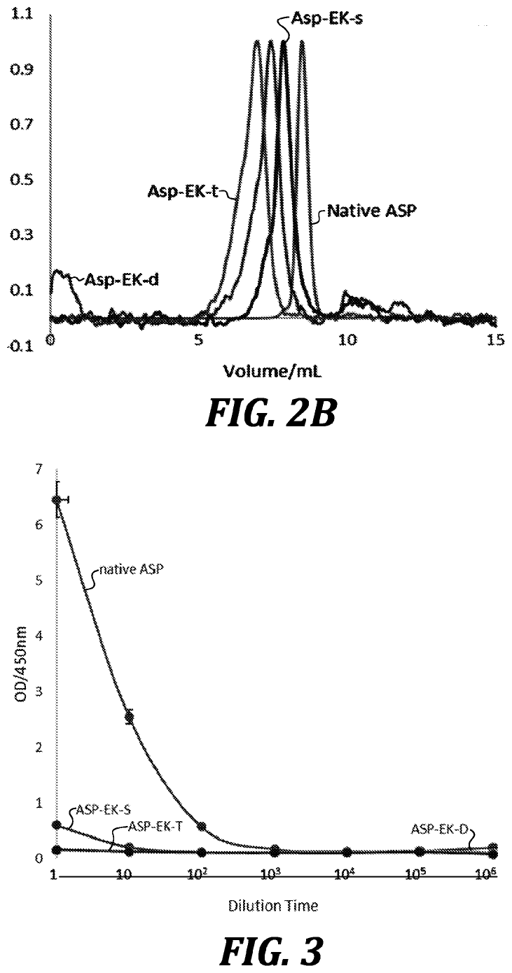

The EK-Asparaginase (EK-ASP) conjugate was prepared using lysine amplification method. Amines on native ASP was first activated by a maleimide-NHS bifunctional crosslinker (N-maleimidoacetoxysuccinimide ester, AMAS). Two equivalents of AMAS crosslinker to amine groups were dissolved in DMSO (20 mg/ml) and added dropwise into ASP solution (2 mg/ml in PBS buffer, pH 7.4). Following half hour of stirring, the reaction mixture was ultra-centrifuged for 5 times against fresh MOPS buffer (0.1M, pH7.0) in a protein concentrator tube (MW cutoff: 30 k Da) to remove unreacted AMAS and any possible small molecule impurities. Residual solution containing AMAS activated ASP was then combined with (EK)₁₀-C stock solution (10 mass-fold excess to ASP, in MOPS buffer) to initiate the first EK layer modification. The conjugation reaction was kept overnight at 4° C. and same ultra-centrifuge step was performed to remove excess EK peptide. Purified ASP-EK-Single Layer (ASP-EK-s) conjugate was stored at 2 mg/ml in 4° C. fridge for further characterization and next conjugation steps. ASP-EK-Double Layer (ASP-EK-d) formulation was prepared by introducing (EK)₁₀-C peptide to the lysines amplified by 1st EK layer. A same AMAS activation followed by EK modification procedure was performed and the resulting ASP-EK-d conjugate was purified by ultra-centrifuge (MW cutoff: 100 k). By the same method, ASP-EK-Triple Layer (ASP-EK-t) formulation was prepared and purified. The preparation process is shown in FIG. 2.

Gel Permeation Chromatography (GPC) was performed to characterize the size differences of ASP-EK-S, ASP-EK-D, and ASP-EK-T products. Also, to obtain the final fully covered ASP-EK-T conjugate products, Hydrophobic Interaction Column (HIC) were used to eliminate those not fully encapsulated.

Specific Binding Affinity Characterization

In order to demonstrate that the three-layer zwitterionic polypeptide-protein conjugates offer significant shielding effects, in vitro ELISA testing was carried out. Generally, streptavidin-coated 96-wells were used as the template, anti-ASP antibody was employed as the first antibody, and then ASP-EK formulation solution were added in wells. After washing, the HRP-conjugated anti-ASP antibody was introduced as second antibody. Thus, OD values generated by HRP/TMB reaction was used to evaluate the binding affinity between ASP and corresponding antibody. Higher binding affinity indicates a lower surface shielding capability. Overall, it was concluded that the increase of EK layer on ASP surface significantly shielded ASP from specific antibody binding. To be specific, compared to the native ASP which has a detection limit of 10³ pmol/mL, the single layer conjugate significantly reduces the binding affinity with a detection limit of 10⁵ pmol/mL, but still has a tinny of binding ones. However, the second and third layer almost fully cover the binding episodes of ASP, indicating a satisfactory sheltering effect under mild conditions.

FIG. 2 compares in vitro anti-asparaginase antibody binding affinity of native ASP, ASP-EK-S, ASP-EK-D, and ASP-EK-T (the original concentration is 1 μmol/mL).

Non-Specific Binding Affinity Characterization

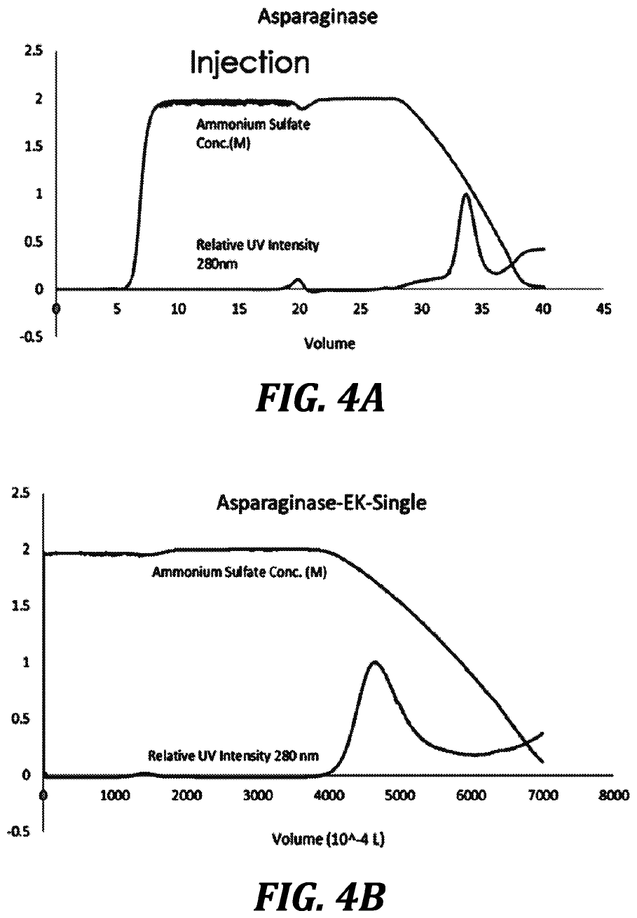

Despite the size increase, there remain a few conjugates that were not fully packed. Therefore, Hydrophobic Interaction Chromatography (HIC) was employed to separate and purify the fully packed conjugates while maintaining biological activity. Once dissolved in the high-salt buffer, the solvation of sample solutes was reduced, and the hydrophobic regions that became exposed were attached to the medium. A series of decreasing salt gradient Tris-HCl buffers were then applied to elute samples from the column, and the fully covered samples were washed out and collected. The HIC results are shown in FIGS. 4A-4D.

Compared to the native ASP, which was eluted at 1.13M concentration, the hyperbranched conjugate shows two peaks. The left one represents the fully covered conjugate, it just goes out with the loading buffer, with no non-specific binding affinity. The other one is eluted at 1.76 M concentration, which shows a litter non-specific binding. Table 1 shows the sample elution gradients, and the three-layer conjugate has the best results.

FIGS. 4A-4D compare the HIC results of native ASP (4A), ASP-EK-S (4B), ASP-EK-D (4C), and ASP-EK-T (4D).

TABLE 1
Sample elution gradients

| Asparaginase | Ammonium Sulfate |
|---|---|
| Formulation | Concentration at Elution |
| Native ASP | 1.13M |
| ASP-EK-s | 1.71M |
| ASP-EK-d | 1.73M |
| ASP-EK-t | 2M (unbound), 1.76M |

In Vivo Pharmacokinetics and Immunogenicity Study of Hyperbranched ASP-EK-T Conjugate

All animal experiments adhered to federal guidelines and were approved by the University of Washington Institutional Animal Care and Use Committee (IACUC). Animals were randomized to treatment groups at the beginning of each study and a sample size of five animals per group was used. C57LB/6 mice (male, body weight 20-25 g) were purchased from Jackson Laboratories.

The pharmacokinetics (PK) of native asparaginase and hyperbranched ASP-EK-t conjugate were studied using two groups of mice. Intravenous (IV) administrations were performed at the first day of each week for consecutive three weeks. 50 L 12.5 mU/mL ASP samples were administered via a tail vein injection method. Blood samples were then collected at 5 min, 1, 4, 8, and 24 hr time points, respectively, relative to the injection time. Blood was collected at day 21 and was used for immunogenicity study (IgM and IgG antibody detection) by indirect ELISA. Also, the enzyme contents in blood serum were estimated by using an Asparaginase Activity Assay Kit.

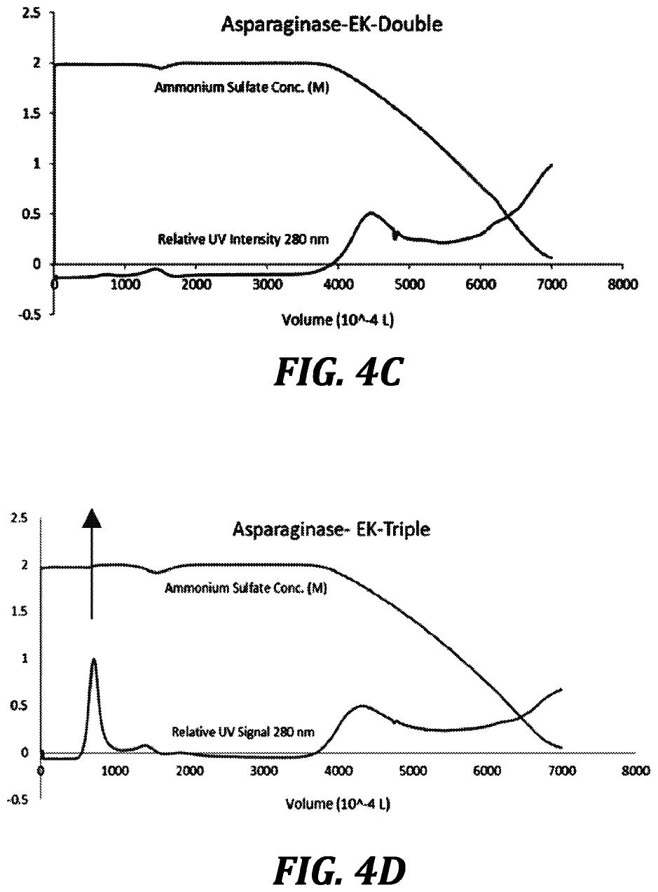

The PK results of both the native ASP group and the ASP-EK-T group are shown in FIGS. 5A-5C. Overall, the ASP-EK-t conjugate significantly outperformed the native ASP and sustained bioactivity for longer periods post-injection. To be specific, the ASP-EK-T conjugate maintained superior circulation time even after the third injection, while the native ASP experienced an obvious accelerated blood clearance. Meanwhile, there is no ABC effect observed in the ASP-EK-t group. The extended and unchanged circulation time of ASP-EK-t conjugate after triple administration reveals the non-fouling property and the size increase, which together evade the fast clearance by immune and renal system.

FIGS. 5A-5C compare PK profiles of ASP formulations 1^{st} dose (5A), 2^{nd} dose (5B), and 3^{rd} dose (5C).

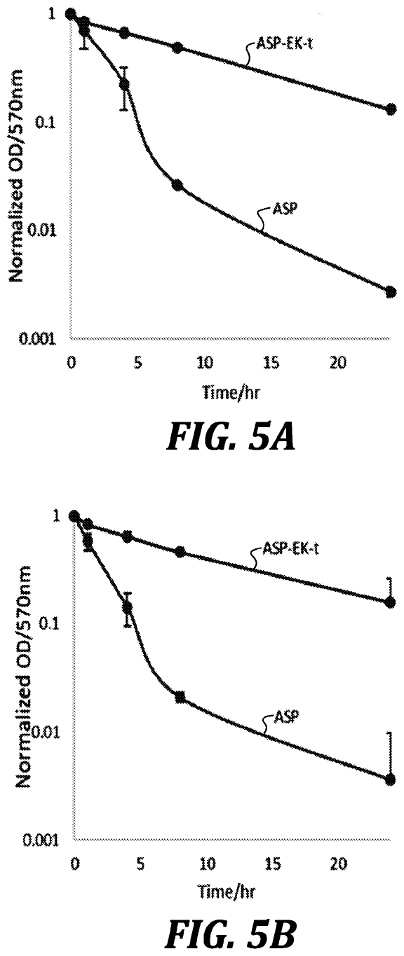

The indirect ELISA assays for IgG testing are shown in FIG. 6. The anti-ASP titer in the ASP-EK-t mice group is much lower than the native ASP group, which demonstrated that the immunogenic episodes were fully shielded by the polypeptide. Also, the super low anti-EK titer in the ASP-EK-t mice group indicates that the synthesized polypeptide is of super low immunogenicity even under complex environments.

FIG. 6 compares detection of week 3 anti-ASP and anti-EK IgG antibodies in mice serum.

### Example 2

The Preparation of a Representative Multi-Layer Bioconjugate: Asp-EK-PCB

In this example, the preparation of a representative multi-layer polymer bioconjugate, Asp-EK-PCB, is described.

Synthesis of (EK)₃-C Peptide

(EK)₃-C was synthesized by Fmoc Solid Phase Peptide Synthesis (SPPS) on Liberty Blue Automated Microwave Assisted Peptide Synthesizer (CEM). Sequence synthesis scale was set at 2.5 mmol on Rink amide MBHA resin (0.6 meq/g substitution). Deprotection was performed in 20% piperidine/DMF solution with machine default microwave conditions. Coupling reactions were performed in the presence of a 5-fold molar excess of reagents [0.2 M amino acid solution (in DMF) with 0.5M DIC (in DMF) and 1.0 M Oxyma (in DMF)] by using 2.5 mmol coupling cycle method provided from CEM. Cleavage was performed using 20 ml of cocktail. (TFA/phenol/water/thioanisole/EDT; 82.5/5/5/5/2.5) for 180 min at room temperature. Following cleavage, (EK)₃-C—NH₂ was precipitated out and washed with ice-cold anhydrous ethyl ether. (EK)₃-C—NH₂ was further purified by recrystallization.

Preparation of PCB-SH Polymers

4 g of amine group terminated PCB polymer (PCB-NH₂, Mw: 10 k) was activated by Traut's reagent (50 mg) to obtain PCB-SH. Two reactants were kept stirring in 500 mL HEPES buffer for 1 h. Unreacted Traut's reagent was removed by a desalting column with HEPES as an operation buffer just before the conjugation step. The polymer solution stock at 100 mg/ml in HEPES was directly used in the next step.

Preparation of Asp-PCB Conjugates

Amines on native ASP was first activated by a maleimide-NHS bifunctional crosslinker (N-maleimidoacetoxysuccinimide ester, AMAS). Two equivalents of AMAS crosslinker to amine groups were dissolved in DMSO (20 mg/ml) and added dropwise into ASP solution (2 mg/ml in PBS buffer, pH 7.4). Following half hour of stirring, the reaction mixture was ultra-centrifuged for 5 times against fresh MOPS buffer (0.1M, pH7.0) in a protein concentrator tube (MW cutoff: 30 k Da) to remove unreacted AMAS and any possible small molecule impurities. Residual solution containing AMAS activated ASP was then combined with PCB-SH stock solutions and kept stirring at 4° C. for 4 h. The conjugation reaction was kept overnight at 4° C. Asp-PCB conjugates were purified by ultra-centrifuge (Mw cutoff: 100 k).

Preparation of Asp-EK Conjugates

Amines on native ASP was first activated by a maleimide-NHS bifunctional crosslinker (N-maleimidoacetoxysuccinimide ester, AMAS). Two equivalents of AMAS crosslinker to amine groups were dissolved in DMSO (20 mg/ml) and added dropwise into ASP solution (2 mg/ml in PBS buffer, pH 7.4). Following half hour of stirring, the reaction mixture was ultra-centrifuged for 5 times against fresh MOPS buffer (0.1M, pH7.0) in a protein concentrator tube (MW cutoff: 30 k Da) to remove unreacted AMAS and any possible small molecule impurities. Residual solution containing AMAS activated ASP was then combined with (EK)₃-C stock solution (10 mass-fold excess to ASP, in MOPS buffer) to initiate the first EK layer modification. The conjugation reaction was kept overnight at 4° C. and same ultra-centrifuge step was performed to remove excess EK peptide. Purified ASP-EK-Single Layer (ASP-EK-s) conjugate was stored at 2 mg/mL in 4° C. refrigerator for next conjugation steps.

Preparation of Asp-EK-PCB Conjugate

Amines on native Asp-EK was first activated by a maleimide-NHS bifunctional crosslinker (N-maleimidoacetoxysuccinimide ester, AMAS). Two equivalents of AMAS crosslinker to amine groups were dissolved in DMSO (20 mg/mL) and added dropwise into ASP solution (2 mg/mL in PBS buffer, pH 7.4). Following 30 min stirring, the reaction mixture was ultra-centrifuged for 5 times against fresh MOPS buffer (0.1M, pH 7.0) in a protein concentrator tube (MW cutoff: 30 k Da) to remove unreacted AMAS and any possible small molecule impurities. Activated Asp-EK conjugates and PCB-SH stock solutions were combined and kept stirring at 4° C. for 4 h. Excess polymer and unreacted protein was removed by Diafiltration (Mw cutoff: 100 k, KR2i system, Spectrum).

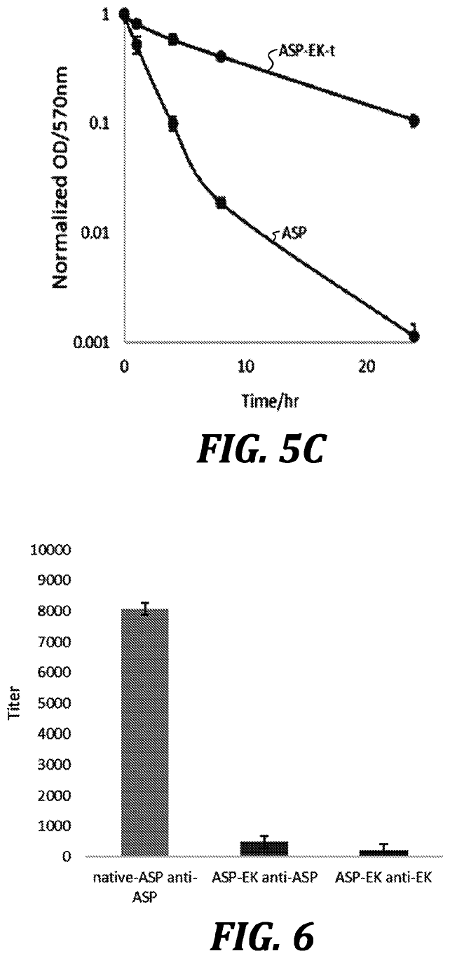

As shown in FIG. 7, multilayer Asp-EK-PCB conjugates showed significant size improvement compared to Asp-PCB single layer conjugates. It is noted that the size distribution of Asp-EK-PCB conjugates were also narrowed, indicating the improved polymer density of Asp conjugates.

### Example 3

The Preparation of a Representative Multi-Layer Bioconjugate: Uricase-EK-PCB

In this example, the preparation of a representative multi-layer polymer bioconjugate, Uricase-EK-PCB, is described.

Synthesis of (EK)₃-C Peptide

(EK)₃-C was synthesized by Fmoc Solid Phase Peptide Synthesis (SPPS) on Liberty Blue Automated Microwave Assisted Peptide Synthesizer (CEM). Sequence synthesis scale was set at 2.5 mmol on Rink amide MBHA resin (0.6 meq/g substitution). Deprotection was performed in 20% piperidine/DMF solution with machine default microwave conditions. Coupling reactions were performed in the presence of a 5-fold molar excess of reagents [0.2 M amino acid solution (in DMF) with 0.5M DIC (in DMF) and 1.0 M Oxyma (in DMF)] by using 2.5 mmol coupling cycle method provided from CEM. Cleavage was performed using 20 ml of cocktail (TFA/phenol/water/thioanisole/EDT; 82.5/5/5/5/2.5) for 180 min at room temperature. Following cleavage, (EK)₃-C—NH₂ was precipitated out and washed with ice-cold anhydrous ethyl ether. (EK)₃-C—NH₂ was further purified by recrystallization.

Preparation of Uri-EK Conjugates

Amines on native Uricase (Uri) was first activated by a maleimide-NHS bifunctional crosslinker (N-maleimidoacetoxysuccinimide ester, AMAS). Two equivalents of AMAS crosslinker to amine groups were dissolved in DMSO (20 mg/mL) and added dropwise into Uri solution (2 mg/ml in PBS buffer, pH 7.4). Following 30 min stirring, the reaction mixture was ultra-centrifuged for 5 times against fresh MOPS buffer (0.1M, pH 7.0) in a protein concentrator tube (MW cutoff: 30 k Da) to remove unreacted AMAS and any possible small molecule impurities. Residual solution containing AMAS activated Uri was then combined with (EK)₃-C stock solution (10 mass-fold excess to Uri, in MOPS buffer) to initiate the first EK layer modification. The conjugation reaction was kept overnight at 4° C. and same ultra-centrifuge step was performed to remove excess EK peptide. Purified Uri-EK conjugate was stored at 2 mg/ml in 4° C. refrigerator for next conjugation steps.

Preparation of Asp-EK-PEG Conjugate

Amines on native Uri-EK was first activated by a maleimide-NHS bifunctional crosslinker (N-maleimidoacetoxysuccinimide ester, AMAS). Two equivalents of AMAS crosslinker to amine groups were dissolved in DMSO (20 mg/mL) and added dropwise into Uri-EK solution (2 mg/mL in PBS buffer, pH 7.4). Following 30 min stirring, the reaction mixture was ultra-centrifuged for 5 times against fresh MOPS buffer (0.1M, pH 7.0) in a protein concentrator tube (MW cutoff: 30 k Da) to remove unreacted AMAS and any possible small molecule impurities. Activated Asp-EK conjugates and PEG-SH (Mw 5 k) stock solutions were combined and kept stirring at 4° C. for 4 h. Excess polymer and unreacted protein were removed by Diafiltration (Mw cutoff: 100 k, KR2i system, Spectrum).

### Example 4

The Preparation and Characterization of Representative Polymer Backfilled Bioconjugates: PCB-OPH

In this example, the preparation and characterization of a representative polymer backfilled bioconjugates, PCB-OPH, is described.

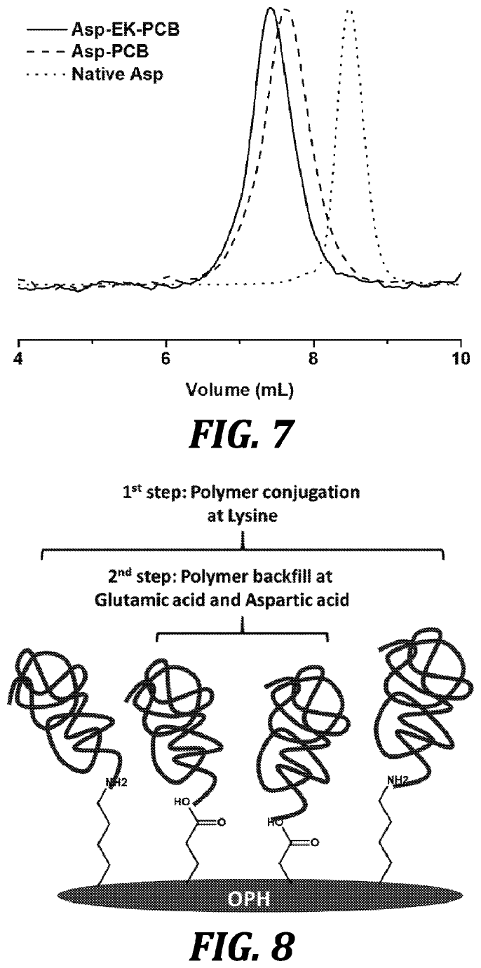

In this example, organophosphorus hydrolase (OPH) was modified to provide a representative polymer backfilled bioconjugates, PCB-OPH. OPH shows a dimer structure after expression in E. Coli. There are only 4 water accessible lysines per monomer, which can be modified with polymers. Common polymer conjugation technology like PEGylation, cannot provide OPH with sufficient steric protection due to the lack of conjugation sites. Besides limited lysines, abundant glutamic acids and aspartic acids distribute evenly on the OPH surface, which provide more conjugation sites to achieve high polymer density. The conjugation includes two main steps as shown in FIG. 8.

Preparation of PCB-SH Polymers

40 g of amine group terminated PCB polymer (PCB-NH₂) was activated by Traut's reagent (500 mg) to obtain PCB-SH. Two reactants were kept stirring in 500 mL HEPES buffer for 1 h. Unreacted Traut's reagent was removed by a desalting column with HEPES as an operation buffer just before the conjugation step. The polymer solution stock at 100 mg/mL in HEPES was directly used in the next step.

Activation of OPH by Maleimide-NHS crosslinker (N-α-maleimidoacetoxysuccinimide ester, AMAS) OPH (1 g) was dissolved in HEPES (200 mL) at 4° C. and combined with crosslinker AMAS (150 mg, 20 mg/mL in dimethyl sulfoxide). The solution was kept stirring at 4° C. for 1 h and unreacted AMAS was removed by a desalting column with HEPES as an operation buffer. The protein solution was concentrated to 20 mg/mL by ultrafiltration.

Preparation of PCB-OPH Conjugate and Purification Process

OPH and PCB-SH stock solutions were combined and kept stirring at 4° C. for 4 h. Excess polymer and unreacted protein were removed by Diafiltration (Mw cutoff: 100 k, KR2i system, Spectrum). Lysines modified PCB-OPH (20 k-OPH) was then used directly in the next backfill step.

PCB Backfill on Carboxyl Groups of PCB-OPH Conjugates

1 g of 20 k-OPH, 40 g of PCB-NH₂ polymers and 2 g of Sulfo-NHS were dissolved in 500 mL of MOPS buffer (pH 7.0). 2 g of EDC in 20 mL MOPS buffer was added the protein solution. After 4 h of stirring at room temperature, the mixed solution was further stirred at 4° C. for 24 h. Excess polymers and small molecular impurities were removed by Diafiltration (Mw cutoff: 100 k, KR2i system, Spectrum). After backfill, the hydrodynamic size of PCB-OPH was significantly enlarged, which indicates the increase of PCB densities.

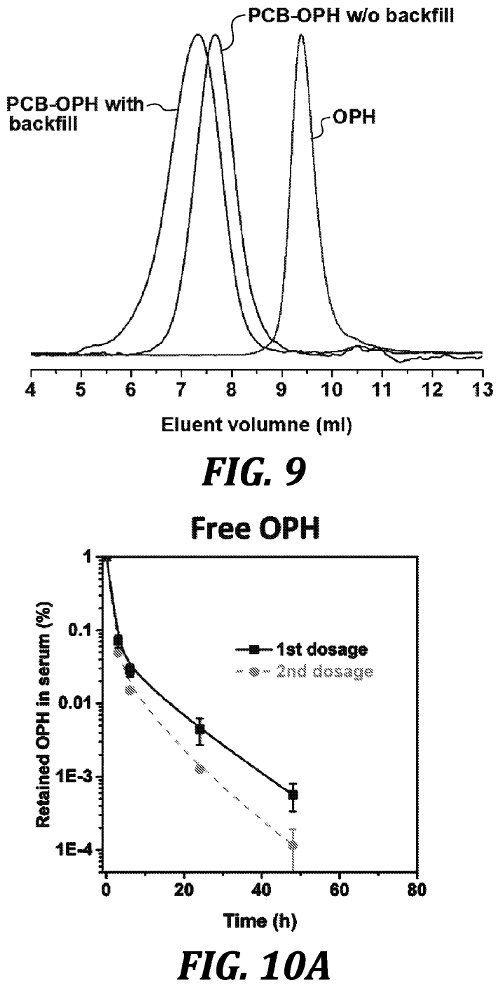

FIG. 9 compares SEC curves of native and PCB modified OPH formulations.

In Vivo Pharmacokinetics and Immunogenicity Study of OPH Conjugate Formulations

All animal experiments adhered to federal guidelines and were approved by the University of Washington Institutional Animal Care and Use Committee (IACUC). Animals were randomized to treatment groups at the beginning of each study and a sample size of six animals per group was used. SD Rats (female, body weight 200-225 g) were purchased from Charles Rivers. 1st and 2nd dosage of OPH formulations were intravenously injected on day 1 and day 15, respectively. Blood was collected and analyzed for PK profiles. After two dosages of stimulation, sera were sampled on day 28 and day 35 to obtain the IgM and IgG levels.

Free OPH showed rapid clearance after 1st injection. Lack of polymer protection, OPH itself could not to maintain long circulation time though its size is greater than renal cutoff limit. The 2nd dosage showed accelerated blood clearance, which is so-called ABC phenomenon, because the generation of anti-OPH antibody. In terms of low PCB covered OPH, it showed remarkable longer circulation time compared to native OPH, but an ABC phenomenon also occurred after the 2nd injection. Backfilled PCB-OPH conjugates showed superior circulation time of 1^{st} dose and unchanged 2^{nd} dose PK profile. Increased PCB density by backfill technology played a crucial role in in vivo drug behavior.

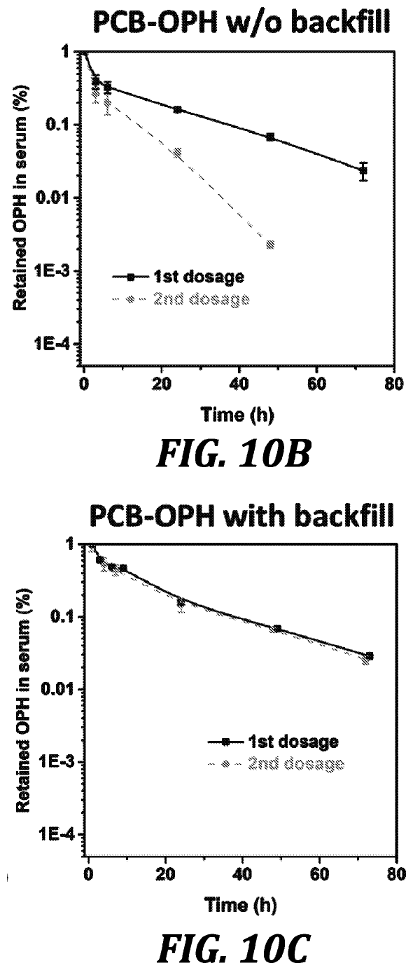

FIGS. 10A-10C compare PK profiles of free OPH (10A), PCB-OPH w/o backfill (10B), and PCB-OPH with backfill (10C).

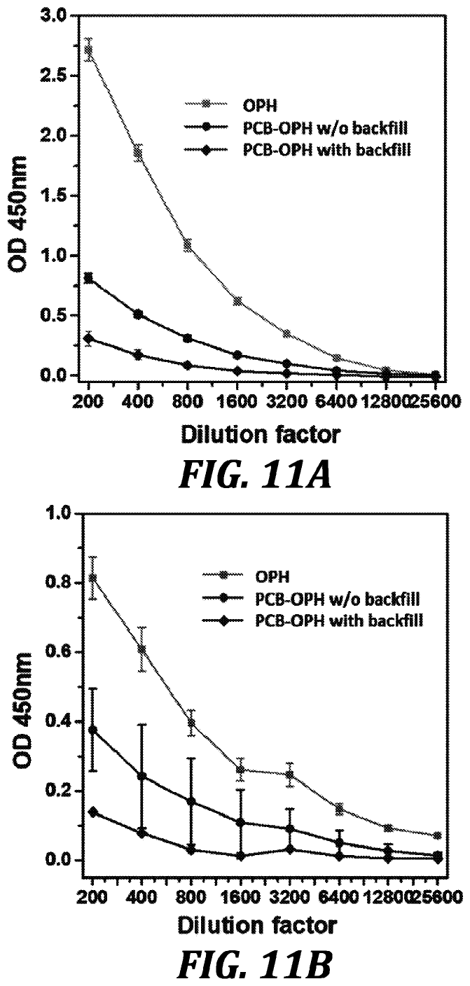

The indirect ELISA assays for IgG and IgM testing are shown in FIGS. 11A and 11B, respectively. The anti-OPH IgM and IgG titers in backfilled PCB-OPH treated Rat group were much lower than native OPH or un-backfilled PCB-OPH treated groups, which demonstrated that the immunogenic episodes were fully shielded by polymer backfill technology.

### Example 5

High Polymer Density of Double Layer OPH-EK-PCB Conjugates with PCB Backfill Synthesis and Characterization of (EK)₁₀-C Polypeptide

(EK)₁₀-C was synthesized by Fmoc Solid Phase Peptide Synthesis (SPPS) on Liberty Blue Automated Microwave Assisted Peptide Synthesizer (CEM). Sequence synthesis scale was set at 2.5 mmol on Rink amide MBHA resin (0.6 meq/g substitution). Deprotection was performed in 20% piperidine/DMF solution with machine default microwave conditions. Coupling reactions were performed in the presence of a 5-fold molar excess of reagents [0.2 M amino acid solution (in DMF) with 0.5M DIC (in DMF) and 1.0 M Oxyma (in DMF)] by using 2.5 mmol coupling cycle method provided from CEM. Cleavage was performed using 20 ml of cocktail (TFA/phenol/water/thioanisole/EDT; 82.5/5/5/5/2.5) for 180 min at room temperature. Following cleavage, (EK)₁₀-C—NH₂ was precipitated out and washed with ice-cold anhydrous ethyl ether. Econo-Pac 10DG Desalting Columns were used to further enhance the purity of the products.

Preparation of PCB-SH Polymers

40 g of amine group terminated PCB polymer (PCB-NH₂) was activated by Traut's reagent (500 mg) to obtain PCB-SH. Two reactants were kept stirring in 500 mL HEPES buffer for 1 h. Unreacted Traut's reagent was removed by a desalting column with HEPES as an operation buffer just before the conjugation step. The polymer solution stock at 100 mg/mL in HEPES was directly used in the next step.

Preparation of OPH-EK Conjugates

Amines on native OPH was first activated by a maleimide-NHS bifunctional crosslinker (N-maleimidoacetoxysuccinimide ester, AMAS). Two equivalents of AMAS crosslinker to amine groups were dissolved in DMSO (20 mg/mL) and added dropwise into OPH solution (2 mg/ml in HEPES buffer, pH 7.2). Following 30 min stirring, the reaction mixture was ultra-centrifuged for 5 times against fresh HEPES buffer (0.1M, pH 7.2) in a protein concentrator tube (MW cutoff: 30 k Da) to remove unreacted AMAS and any possible small molecule impurities. Residual solution containing AMAS activated OPH was then combined with (EK)₁₀-C stock solution (10 mass-fold excess to OPH, in HEPES buffer) to initiate the EK layer modification. The conjugation reaction was kept overnight at 4° C. and same ultra-centrifuge step was performed to remove excess EK peptide. Purified OPH-EK conjugate was stored at 2 mg/ml in 4° C. refrigerator for next conjugation steps.

Preparation of OPH-EK-PCB Conjugate

Amines on native OPH-EK was first activated by a maleimide-NHS bifunctional crosslinker (N-maleimidoacetoxysuccinimide ester, AMAS). Two equivalents of AMAS crosslinker to amine groups were dissolved in DMSO (20 mg/mL) and added dropwise into OPH-EK solution (2 mg/mL in HEPES buffer, pH 7.2). Following 30 min stirring, the reaction mixture was ultra-centrifuged for 5 times against fresh HEPES buffer (0.1M, pH 7.2) in a protein concentrator tube (MW cutoff: 30 k Da) to remove unreacted AMAS and any possible small molecule impurities. Activated OPH-EK conjugates and PCB-SH stock solutions were combined and kept stirring at 4° C. for 4 h. Excess polymer and unreacted protein were removed by Diafiltration (Mw cutoff: 100 k, KR2i system, Spectrum).

PCB Backfill on Carboxyl Groups of PCB-OPH Conjugates

1 g of OPH-EK-PCB, 40 g of PCB-NH₂ polymers and 2 g of Sulfo-NHS were dissolved in 500 mL of MOPS buffer (pH 7.0). 2 g of EDC in 20 mL MOPS buffer was added the protein solution. After 4 h of stirring at room temperature, the mixed solution was further stirred at 4° C. for 24 h. Excess polymers and small molecular impurities were removed by Diafiltration (Mw cutoff: 100 k, KR2i system, Spectrum).

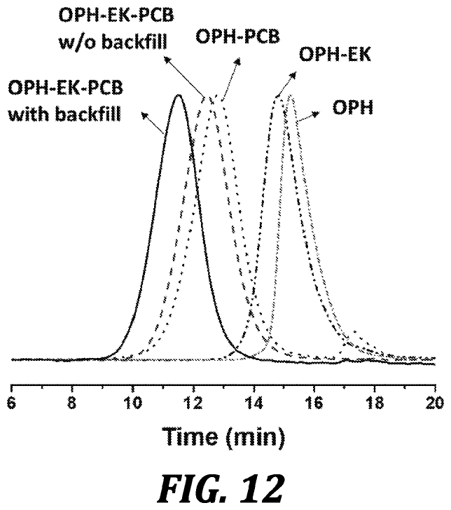

As shown in FIG. 12, every conjugation step increased the size of OPH formulations. The double layer OPH-EK-PCB conjugates plus PCB backfill achieved highest polymer coverage, indicating the combination of multilayer and backfill method significantly increased polymer density.

While illustrative embodiments have been illustrated and described, it will be appreciated that various changes can be made therein without departing from the spirit and scope of the invention.

1. A bioconjugate, comprising a biomolecule having first polymers covalently coupled the biomolecule and second polymers covalently coupled to at least a portion of the first polymers.

2. The bioconjugate of claim 1, wherein the first polymers form a first layer surrounding the biomolecule.

3. The bioconjugate of claim 1, wherein the second polymers form a second layer surrounding the biomolecule.

4. The bioconjugate of claim 1, wherein the biomolecule is a protein, a glycoprotein, a proteoglycan, a lipid, a nucleic acid, a cell, a virus, or a bacterium.

5. The bioconjugate of claim 1, wherein the first and second polymers are independently zwitterionic polymers or peptides.

6. The bioconjugate of claim 1, wherein the first and second polymers are independently EK-containing peptides.

7-8. (canceled)

9. The bioconjugate of claim 6, wherein the EK-containing peptides comprise (EK)ₙ peptides where n is from 1 to about 50.

10. The bioconjugate of claim 1, wherein the first polymers are EK-containing peptides and the second polymers are EK-containing peptides or zwitterionic polymers.

11. (canceled)

12. The bioconjugate of claim 1, wherein the first and second polymers are independently peptides that include one or more amino acid residues selected from lysine, glutamic acid, aspartic acid, cysteine, histidine, serine, threonine, tyrosine, tryptophan, and proline residues.

13. (canceled)

14. The bioconjugate of claim 1, wherein the first polymers are non-peptides polymers that include one or more functional groups selected from amine, carboxylic acid, thiol, maleimide, carbon-carbon double bond, carbon-carbon triple bond, and azido functional groups.

15-18. (canceled)

19. The bioconjugate of claim 1, wherein the second polymers are non-peptides polymers selected from the group consisting of poly(carboxybetaine) (PCB), poly(sulfobetaine) (PSB), poly(2-methacryloyloxyethyl phosphorylcholine) (PMPC), poly(tetramethylamine oxide) (TMAO), poly(2-oxazoline) (POZ), poly(N-(2-hydroxypropyljmethacrylamide) (polyHPMA), and polyethylene glycol (PEG) polymers.

20-21. (canceled)

22. A method for making a multi-layer bioconjugate of claim 1, comprising:
(a) covalently coupling first polymers to a biomolecule to provide a biomolecule having a first polymer layer surrounding the biomolecule; and
(b) covalently coupling second polymers to at least a portion of the first polymers of the first polymer layer to provide a biomolecule having a second polymer layer surrounding the biomolecule.

23-24. (canceled)

25. A bioconjugate, comprising:
(a) biomolecule having one or more first reactive groups and one or more second reactive groups, wherein the first and second reactive groups are different;
(b) one or more first polymers covalently coupled to the first reactive groups; and
(c) one or more second polymers covalently coupled to the second reactive groups.

26. The bioconjugate of claim 25, wherein the biomolecule is a protein, a glycoprotein, a proteoglycan, a lipid, a nucleic acid, a cell, a virus, or a bacterium.

27. The bioconjugate of claim 25, wherein first and second reactive groups are independently selected from the group consisting of amine, carboxylic acid, thiol, maleimide, carbon-carbon double bond, carbon-carbon triple bond, and azido groups.

28-30. (canceled)

31. The bioconjugate of claim 25, wherein first reactive groups are selected from amine groups and the second reactive groups are selected from carboxylic acid (or carboxylate) groups.

32. The bioconjugate of claim 25, wherein the first and second polymers are independently selected from the group consisting of poly(carboxybetaine) (PCB), poly(sulfobetaine) (PSB), poly(2-methacryloyloxyethyl phosphorylcholine) (PMPC), poly(tetramethylamine oxide) (TMAO), poly(2-oxazoline) (POZ), poly(N-(2-hydroxypropyl)methacrylamide) (polyHPMA), and polyethylene glycol (PEG) polymers.

33-38. (canceled)

39. A method for making a bioconjugate of claim 25, comprising:
(a) covalently coupling one or more first polymers to a biomolecule having one or more first reactive groups and one or more second reactive groups, wherein the first and second reactive groups are different, and wherein the first polymers are covalently coupled to the first reactive groups; and
(b) covalently coupling one or more second polymers to the second reactive groups to provide a bioconjugate having first polymers and second polymers covalently coupled to the biomolecule.

40. (canceled)

41. A bioconjugate, comprising
(a) biomolecule having one or more first reactive groups and one or more second reactive groups, wherein the first and second reactive groups are different;
(b) first polymers covalently coupled to the first reactive groups;
(c) second polymers covalently coupled to at least a portion of the first polymers and;
(d) one or more third polymers covalently coupled to the second reactive groups.

42-45. (canceled)

46. A method for making a bioconjugate of claim 41, comprising:
(a) covalently coupling one or more first polymers to a biomolecule having one or more first reactive groups and one or more second reactive groups, wherein the first and second reactive groups are different, and wherein the first polymers are covalently coupled to the first reactive groups;
(b) covalently coupling one or more second polymers to at least a portion of the first polymers; and
(c) covalently coupling one or more third polymers to the second reactive groups to provide a bioconjugate having first polymers, second polymers, and third polymers covalently coupled to the biomolecule.
